(19) United States
(12) Patent Application Publication
(10) Pub. No.: US 2002/0172948 A1
(43) Pub. Date: Nov. 21, 2002

(54) Method and system for nucleic acid sequencing

(76) Inventor: Mark W. Perlin, Pittsburgh, PA (US)

Correspondence Address: Ansel M. Schwartz, Attorney at Law, Suite 304, 201 N. Craig Street, Pittsburgh, PA, 15213, US

(21) Appl. No.: 09/849,809

(22) Filed: May 4, 2001

(51) Int. Cl. G01V 1/40; C12Q 1/68; G01V 3/18; C12P 19/34

(52) U.S. Cl. 435/6; 435/91.2; 702/20

(57) ABSTRACT

A method of nucleic acid sequencing comprising the steps (a) amplifying a nucleic acid sample to produce an amplified DNA product; (b) extending a sequencing primer bound to the DNA product in the presence of terminating nucleotide analogs to produce a collection of labeled nucleic acid products; (c) detecting a total amount of label present in the collection to produce a measurement; and (d) combining a plurality of measurements to determine DNA sequence information about the sample. A system for nucleic acid sequencing which uses terminating nucleotide analogs to quantitatively determine fragment length and sequence information.

### FIELD OF THE INVENTION

The present invention pertains to a process for determining information about the sequence of a DNA molecule. More specifically, the present invention is related to performing experiments that produce quantitative data, and then using these data to determine DNA sequence information, such as DNA molecule length or nucleotide composition. The invention also pertains to systems related to this sequence information.

### BACKGROUND OF THE INVENTION

The high cost of genetic information limits current research and expectations for clinical application. The total data acquisition cost for a DNA fragment sizing experiment is about one dollar for each genotype—a dollar per bit. Similar costs are incurred with gene sequencing for mutation analysis. For large-scale efforts (e.g., gene discovery or population screening), these costs all but prohibit rapid progress. In cancer genetics, this high cost-per-bit limits the widespread use of assays for genetic polymorphism, microsatellite instability (MI), loss of heterozygosity (LOH), mutation detection, and other important genetic events.

A major cost factor in DNA sizing assays is their current reliance on one-dimensional (1-D) size separation technologies. These assays use the “lane” as the readout pathway. However, there are practical limitations on the degree of multiplexing within each lane, as well as on the number of lanes per run. Recently, DNA arrays comprised of a 2-D arrangement of 0-D dots have been used to replace certain DNA size separation assays. By packing in many dots, these arrays can provide a 100-fold increase in data density, relative to lane-based methods. When the biochemistry can be performed directly on the array surface, this density can translate into an equivalent reduction in the genetic cost-per-bit.

The invention described herein is a novel method for characterizing DNA fragments, dubbed “DNA transform sequencing.” The described invention exploits the chemistry of DNA sequencing to obtain numerical values that provide information about the sequence. It can be used to size DNA fragments in a 0-D “lane-free” format, without performing a size separation. It can also be used for DNA sequencing. The method (1) enables massively parallel array-based DNA analysis, (2) decouples the biochemistry from the signal detection, and (3) may provide a 100-fold cost reduction relative to current assays in certain applications.

This specification describes a robust assay for DNA transform sequencing that includes the following components:

(a) chemistry, including polymerase, labels, template, and dNTP analogs;

(b) substrate, providing a parallel, scalable DNA support format;

(c) detection, measuring signal intensity without performing DNA separation; and

(d) analysis, determining DNA sequence information by transforming the signal.

Useful applications of the DNA transform sequencing invention include:

(a) sizing, including STR genetic markers;

(b) sequencing, such as mutation detection;

(c) cancer, particularly DNA polymorphism assays; and

(d) genetics, including diagnosis and human identity.

The array-based embodiment of the invention for DNA fragment analysis and short-range sequencing enables mass screening of (clinical or research) samples at a very low cost. Useful research and clinical applications include microsatellite analysis (for MI and LOH tumor monitoring), disease susceptibility genetic markers, and mutation detection of disease genes.

Another useful embodiment of the invention is in a scalable DNA microarray format. Such arrays provide a 100-fold or greater reduction in the cost-per-bit of genetic assays. This enables low-cost high-information genetic profiling, with applications to (1) determining population-wide genetic predisposition, (2) individually customized disease prevention, diagnosis and therapy, and (3) effective genetic monitoring of healthy and disease states, including tumors.

### SUMMARY OF THE INVENTION

A method of nucleic acid sequencing comprising the steps (a) amplifying a nucleic acid sample to produce an amplified DNA product; (b) extending a sequencing primer bound to the DNA product in the presence of terminating nucleotide analogs to produce a collection of labeled nucleic acid products; (c) detecting a total amount of label present in the collection to produce a measurement; and (d) combining a plurality of measurements to determine DNA sequence information about the sample. A method as described wherein each measurement of a label corresponds to an amount of terminating nucleotide. A method as described wherein the DNA sequence information corresponds to a length of the DNA sequence. A method as described wherein the DNA sequence information corresponds to a plurality of bases in the DNA sequence.

### BRIEF DESCRIPTION OF THE DRAWINGS

In the accompanying drawings, the preferred embodiment of the invention and preferred methods of practicing the invention are illustrated in which:

FIG. 1 shows the relative amounts of terminated fragments produced for the DNA sequence “ACGTAAGTAAAT” in the presence of ddNTP, with extension probability p=0.8. The bars represent the four different DNA bases A, C, G and T.

FIG. 2 shows the cluster classification with two Laplace coefficients, p=0.5 and p=0.25. Each axis corresponds to one of the coefficients. Legend: one fragment (circle), two fragments (star).

FIG. 3 shows the ABI/310 readout of the sequence extension of the (CA)₁G template using 100 pM of ddATP relative to 50 pM of dATP. The 5′ strand end label (NED) shows that the two peaks have roughly equal height.

FIG. 4 shows the ABI/310 readout of the sequence extension of the (CA)₂G template using 100 pM ddATP and 50 pM dATP.

FIG. 5 shows the ABI/310 readout of the sequence extension of the combined (CA)₁G and (CA)₂G templates using 100 pM ddATP and 50 pM dATP. This signal combines the signals from the individual alleles.

FIG. 6 shows tables of observed data. (a) In this table, each column is the signature observed for a unique pair of DNA fragment lengths. (b) In this table, the pairwise Euclidean distances between the genotype signatures. (c) In this table, for each heterozygotic allele pair, its observed signature is shown (left) together with the average (right) of the two observed signatures of its component alleles.

### DESCRIPTION OF THE PREFERRED EMBODIMENT

### I. DNA Fragment and Sequence Analysis

Automated DNA analysis by electrophoretic separation has been one of the enabling foundations of the genomics revolution. In particular, these separations permit the sizing of DNA fragments, and the determination of DNA sequences.

### Polymorphism

Genetic variation is a key means of finding disease genes, monitoring tumors, and determining genetic predisposition to disease. In the near future, a detailed profile of an individual's polymorphisms (relative to those of his family and population) will help prevent disease by applying genetic knowledge to directed diagnosis and treatment. Indeed, the field of pharmacogenetics is predicated on the eventual customization of pharmacological therapies to individual genetic variation.

Geneticists assay polymorphism in several ways. In non-coding DNA, length variations are both abundant and easily assayable. Length polymorphisms include restriction fragment length polymorphisms (RFLP), amplified fragment length polymorphisms (AFLP), variable nucleotide tandem repeats (VNTR), and short tandem repeats (STR), including the CA-repeat microsatellite polymorphisms (Weber, J., and May, P., 1989, “Abundant class of human DNA polymorphisms which can be typed using the polymerase chain reaction,” Am. J. Hum. Genet., 44: 388-396), incorporated by reference, and tetranucleotide repeat markers. Length polymorphisms are measured by sizing on 1-D electrophoretic lanes. The balletic single nucleotide polymorphisms (SNPs) have less genetic power, but have been developed in anticipation of more scalable 2-D array technologies.

For a given STR marker of an individual, each chromosome contributes one fragment length allele. PCR amplification of the marker amplifies these fragments, so the observed electrophoretic signal contains peaks corresponding to the DNA fragment lengths. There are over 10,000 genetically mapped STRs (Gyapay, G., Morissette, J., Vignal, A., Dib, C., Fizames, C., Millasseau, P., Marc, S., Bernardi, G., Lathrop, M., and Weissenbach, J., 1994, “The 1993-94 Genethon Human Genetic Linkage Map,” Nature Genetics, 7(2): 246-339), incorporated by reference. The STR length polymorphisms can be automatically assayed by electrophoretic separation on fluorescent DNA sequencers (Ziegle, J. S., Su, Y., Corcoran, K. P., Nie, L., Mayrand, P. E., Hoff, L. B., McBride, L. J., Kronick, M. N., and Diehl, S. R., 1992, “Application of automated DNA sizing technology for genotyping microsatellite loci,” Genomics, 14: 1026-1031), incorporated by reference.

In DNA coding regions, mutations can be detected by sequencing the mutation for an individual patient. Most DNA sequencing currently entails generating a 1-D lane of data by electrophoretic separation. However, the actual sequence variation is most often contained within a very short gene subsequence.

### Cancer Applications

STRs are invaluable biomarkers for understanding cancer. They can be used as linked genetic markers for a trait, and microsatellites can show the progression of tumors, as follows:

(a) Somatic deletions of chromosomal regions that contain tumor suppressor genes are helpful in mapping tumor-specific genes and in monitoring patients with specific tumors. These somatic deletions can be detected as a loss of heterozygosity (LOH) through microsatellite analysis of tumor tissues.

(b) Mismatch repair genes help eliminate PCR errors during DNA replication. Defects in these DNA repair genes can be detected via microsatellite instability (MI)—a change in the allele patterns of a tumor relative to normal tissue. MI is also called replication error (RER).

With the advent of fluorescent-based microsatellite genotyping, there has been considerable interest in automating the detection of LOH (Canzian, F., Salovaara, A., Kristo, P., Chadwick, R. B., Aaltonen, L. A., and de la Chapelle, A., 1996, “Semiautomated assessment of loss of heterozygosity and replication error in tumors,” Cancer Research, 56: 3331-3337), and MI (Cawkwell, L., Ding, L., Lewis, F. A., Martin, I., Dixon, M. F., and Quirke, P., 1995, “Microsatellite instability in colorectal cancer: improved assessment using fluorescent polymerase chain reaction,” Gastroenterology, 109: 465-471), incorporated by reference. Tumor studies on fluorescent automated DNA sequencers have demonstrated that reproducible quantitative analysis is possible.

Gene mutations in coding regions are a large source of genetic variation. Some disease-related genes, such as BRCA1 for breast and ovarian cancers (Friedman, L., Ostermeyer, E., Szabo, C., Dowd, P., Lynch, E., Rowell, S., and King, M., 1994, “Confirmation of BRCA1 by analysis of germline mutations linked to breast and ovarian cancer in ten families,” Nature Genet., 8(4): 399-404) have mutations that are associated with increased disease risk (Castilla, L., Couch, F., Erdos, M., Hoskins, K., Calzone, K., Garber, J., Boyd, J., Lubin, M., Deshano, M., Brody, L., Collins, F., and Weber, B., 1994, “Mutations in the BRCA1 gene in families with early-onset breast and ovarian cancer,” Nature Genet., 8(4): 387-91; Struewing, J., Brody, L., Erdos, M., Kase, R., Giambarresi, T., Smith, S., Collins, F., and Tucker, M., 1995, “Detection of eight BRCA1 mutations in 10 breast/ovarian cancer families, including 1 family with male breast cancer,” Am. J. Hum. Genet., 57(1): 1-7), incorporated by reference. Sequencing the exons of such cancer genes can help identify patients who would benefit from proactive diagnosis or treatment. To implement population-wide cancer screening programs, inexpensive focused sequencing technologies are useful.

### Sequencing Technologies

Dideoxy terminator sequencing. The classic Sanger sequencing approach (and its derivatives) use dideoxy terminator nucleotide (ddNTP) analogs (Sanger, F., Nicklen, S., and Coulson, A. R., 1977, “DNA sequencing with chain-terminating inhibitors,” Proc Natl Acad Sci USA, 74(12): 5463-5467), incorporated by reference. Whereas a normal deoxy nucleotide (dNTP) permits chain extension, a ddNTP cannot be extended and therefore terminates the sequencing reaction. Adding labeled ddATP to a sequencing reaction, and size separating by electrophoresis, forms a ladder of terminated strands that correspond to just those DNA subsequences which have Adenosine as the last base. Combining four such ladders (one for each labeled ddATP, ddCTP, ddGTP, and ddTTP) will recover the DNA sequence.

1-D electrophoretic readout. Fluorescent gel (PE Biosystems ABI/377, Hitachi FM/BIO) and capillary array (PE Biosystems ABI/3700, Molecular Dynamics MegaBACE) devices automate the size separation of labeled DNA fragments. These DNA sequencing instruments can also be used for determining the lengths of DNA fragments relative to sizing standards. An inherent limitation of this flexible technology is the cost of a full 1-D readout, which is always performed regardless of the desired information content.

Sequencing by hybridization. There are DNA sequencing methods that do not use size separation. One such approach is “sequencing by hybridization” (SBH), which probes arrayed DNA sequences with oligonucleotides in order to ascertain information about the sequence (Drmanac, R., Drmanac, S., Strezoska, Z., Paunesku, T., Labat, I., Zeremski, M., Snoddy, J., Funkhouser, W. K., Koop, B., and Hood, L., 1993, “DNA sequence determination by hybridization: a strategy for efficient large-scale sequencing,” Science, 260: 1649-1652), incorporated by reference. Hyseq's system probes oligos against arrayed samples, whereas Affymetrix' chips (Fodor, S. P. A., Read, J. L., Pirrung, M. C., Stryer, L., Lu, A. T., and Solas, D., 1991, “Light-directed spatially addressable parallel chemical synthesis,” Science, 251: 767-773), incorporated by reference, probe the sample against arrayed oligos. SBH works best with known sequence variations (e.g., gene mutations) for which a set of informative oligos can be manufactured. The gene chips may have less utility when more flexible DNA sequencing is required.

Sequencing by synthesis. Another gel-free approach is adding one base to a nascent DNA strand, detecting which base was added, and then repeating the process (synthesis+detection) until the sequence is determined (Cheeseman, P. C., 1994, “Method for sequencing polynucleotides,” U.S. Pat. No. 5,302,509; filed Feb. 27, 1991, published Apr. 12, 1994), incorporated by reference. There is a new commercial variation in which each step fills in the appropriate nucleotide for its full extent in the template (Ronaghi, M., Karamohamed, S., Pettersson, B., Uhlen, M., and Nyren, P., 1996, “Real-time DNA sequencing using detection of pyrophosphate release,” Anal Biochem, 242(1): 84-9), incorporated by reference. These potentially powerful methods suffer from an instrumentation constraint: the biochemical synthesis and the physical detection must be combined into a single complex DNA sequencing device. Decoupling the two processes might permit the use of simpler off-the-shelf instrumentation, and allow more parallelization at a lower cost.

### II. Human Tumors

Gastrointestinal (GI) tumors have a high incidence in the US population. The NCI SEER program shows that colorectal cancer has a 47 per 100,000 occurrence rate (1973-1991), while esophageal, stomach, pancreatic and liver cancers have a combined 24 per 100,000 occurrence rate.

To illustrate with just one example, the incidence of esophageal adenocarcinoma (EAdCa) in the U.S. is increasing at an exponential rate of 5%-10% per year, a rate virtually faster than that of any other cancer (Pera, M., Cameron, A., Trastek, V., Carpente, H., and Zinsmeister, A., 1993, “Increasing incidence of adenocarcinoma of the esophagus and esophagogastric junction,” Gastroenterology, 104: 510-4.), incorporated by reference. While great advances have been made in the treatment of many cancers, the prognosis for EAdCa remains grim, with an overall five-year survival of only 5%-12% and a median survival of only 7-9 months (Boring, C., Squires, T., and Tong, T., 1993, “Cancer Statistics,” CA Cancer J Clin, 43(1): 7-26), incorporated by reference. This problem may occur in part because EAdCa is often not recognized until the patient presents with symptoms of advanced disease, such as dysphagia, weight loss, or anemia. While the reasons for the dramatic rise in incidence are unknown, it is well established that nearly all EAdCa arise from a premalignant lesion of the esophagus known as Barrett's esophagus (BE) (Hamilton, S., and Smith, R., 1987, “The relationship between columnar epithelial dysplasia and invasive adenocarcinoma arising in Barrett's esophagus,” Am J Clin Pathol, 87: 301-5; Sjogren, R., and Johnson, L., 1983, “Barrett's esophagus: A review,” Amer J Medicine, 74: 313-6), incorporated by reference. It would be useful to accurately identify the subset of patients with premalignant disease (such as BE) that are progressing toward malignant transformation, and provide effective treatment before invasive EAdCa develops. DNA assays that can detect chromosomal or DNA expression abnormalities in BE that leading to deregulated cell growth can help in this early identification.

Objective biomarkers of malignant transformation can focus on key components of the underlying pathologic mechanisms. DNA transform sequencing systems can provide chromosomal assays for tumor systems, including cancers of the gastrointestinal system, reproductive organs, breast, prostate, lung, skin, central nervous system, endocrine system, blood, lymph, and other mammalian cell types. Such applications using the high-throughput DNA transform sequencing invention will rapidly lead to highly informative biomarkers.

### III. Array Technologies

DNA array techologies have been developed to increase the density and parallelization of experiments. There are several types of arrays: microtiter plates, high-density robotically gridded surfaces, and very high-density gridded microarrays. All of these types permit test-tube experiments to be scaled up in ways that reduce considerably the required time, cost, error and effort of DNA experiments.

Physical mapping experiments entail the comparison of one probe against a library of DNA fragments. A high-density, robotically gridded approach was developed to assay 10,000 to 100,000 fragments in one experiment (Maier, E., Hoheisel, J. D., McCarthy, L., Mott, R., Grigoriev, A. V., Monaco, A., Larin, Z., and Lehrach, H., 1992, “Complete coverage of the Schizosaccharomyces pombe genome in yeast artificial chromosomes,” Nature Genetics, 1: 273-277), incorporated by reference. The use of short-range oligonucleotides probes stimulated SBH research for parallel DNA sequencing (Lehrach, H., Drmanac, A., Hoheisel, J., Larin, Z., Lennon, G., Monaco, A. P., Nizetic, D., Zehetner, G., and Poustka, A., 1990, “Hybridization fingerprinting in genome mapping and sequencing,” In “Genetic and Physical Mapping I: Genome Analysis”, Davies, K. E., and Tilghman, S. M., eds., 39-81, Cold Spring Harbor, N.Y.: Cold Spring Harbor Laboratory; Pevzner, P., and Belyi, I., 1997, “Software for DNA sequencing by hybridization,” Comput Appl Biosci, 13(2): 205-10), incorporated by reference. Reversing the roles of probe and sample led to the current oligo chip arrays for DNA sequencing (Fodor, S. P. A., Read, J. L., Pirrung, M. C., Stryer, L., Lu, A. T., and Solas, D., 1991, “Light-directed spatially addressable parallel chemical synthesis,” Science, 251: 767-773), incorporated by reference. Government and industrial support for array technology have helped stimulate rapid growth in this area.

DNA arrays are useful whenever one hybridizes a probe against many DNA targets. The hybridization can simply (but powerfully) compare a labeled probe against the target array, as with gene expression experiments (Schena, M., Shalon, D., Heller, R., Chai, A., Brown, P. O., and Davis, R. W., 1996, “Parallel human genome analysis: microarray-based expression monitoring of 1000 genes,” Proc Natl Acad Sci USA, 93(20): 10614-10619), incorporated by reference. In more complex situations, the hybridization initiates a biochemical reaction, such as single nucleotide extension minisequencing. The possibility of such highly parallelizable array-based assays has accelerated the considerable investment in SNP resources for detecting genetic polymorphism. Indeed, the array possibilities far outweigh the known SNP limitations (low information content, uncertain error detection, unreliabile assays).

This patent application describes the use of arrays for performing a nonstandard DNA sequencing reaction. The invention exploits the major features of DNA array technology, including scalability, parallelization (of both experiment and detection), and miniaturization. This approach requires an assay that can acquire useful sequence information from a 0-D dot. Such a novel and unobvious new assay method is introduced in the Description of the Preferred Embodiment.

### IV. Information Transforms

### Rationale

There are many ways to represent information. “Information transforms” (also called “mathematical transforms”) are useful tools that preserve information between different representations. For example, the DNA sequence

ACGT AAGT AAAT AAAA

can be equivalently represented by four 0/1 sequencing ladders. The “A” ladder is:

1000 1100 1110 1111

The information contained in the four letter sequence is identical to that in the four 0/1 ladders. Indeed, this ladder representation is the basis of Sanger sequencing.

Other information transformations lead to less apparent representations. Such transformations often entail mathematical operations. There are two important features of such transformations:

(1) invertibility: the ability to move easily (e.g., via computer programs) between different representations having identical information content; and

(2) information reduction: the potential for representing information in a simpler way that requires less data, hence fewer experiments.

### Mathematics

As an example of information reduction, consider the well-known Gaussian normal bell-curve distribution. One way to represent this function is by recording its y value for every value of x. In the worst case, this representation would entail recording infinitely many points. Alternatively, one can change the representation of the normal curve by using a Polynomial Transform that determines central moments (Hoel, P.G., 1971, “Introduction to Mathematical Statistics,” New York: John Wiley & Sons), incorporated by reference. In doing so, one finds that just two numbers completely determine the function:

the first coefficient: the mean μ, and

the second coefficient: the variance σ².

The mathematics is very helpful here. It is far more practical to design experiments that estimate two parameters (μ and σ) in the central moment representation, than it would be to try to observe and estimate every point along the frequency curve.

The Fourier Transform (FT) is perhaps the most ubiquitous information transform (Papoulis, A., 1962, “The Fourier Integral and its Applications,” New York: McGraw-Hill), incorporated by reference. The FT transforms signals into their frequency content. Since the FT is invertible, it can also change the frequency spectrum back into the original signal, without losing any information. Such transforms are used by engineers for high-speed data compression (e.g., modems) and by nature for sensory functions (e.g., hearing sound). In medical magnetic resonance imaging (MRI), the image is actually the inverse FT of the acquired data (Kumar, A., Welti, D., and Ernst, R. R., 1975, “NMR Fourier Zeugmatography,” J. Magn. Resonance, 18: 69-83), incorporated by reference.

Another common information transform is the Laplace Transform (LT) (Boyce, W. E., and DiPrima, R. C., 1996, “Elementary Differential Equations and Boundary Value Problems,” 6th Edition Edition. New York: John Wiley & Sons), incorporated by reference. Rather than examining a signal's frequency response, the LT explores how the function responds to varying degrees of damping. That is, each LT coefficient answers the question: if one applies a decay curve (determined by the coefficent) to the signal, how much total signal is measured? The representation comprised of these damping responses is equivalent (in its information content) to the original signal. This LT concept is useful in implementing the DNA transform sequencing method.

### Partial Information

There are times (as with the bell curve example above) when there is far less information in a signal than the original signal representation would suggest. For example, in a fragment analysis of STR data, there are at most two allele sizes. The electropherogram signal may stretch over 50 base pairs (bp), and contain numerous data artifacts (noise, PCR stutter, relative amplification, +1 artifact, and so on). But the information content is still just the two allele sizes. Therefore, in principle, only two data points (in the correct representation) should uniquely determine the genotype.

Similarly, suppose that there are three known mutations in a gene's 500 bp. The DNA sequencer's lane representation permits 4⁵⁰⁰ (˜10³⁰⁰) possible signals in a 500 bp readout. Yet prior knowledge allows that there are only three possible signals, and so (in some proper representation) at most three data points should answer the question.

The DNA transform sequencing invention uses highly adaptable representations in order to greatly reduce the number (and cost) of required experiments,

### V. Some Advantages

The DNA transform sequencing invention can significantly reduce data acquisition costs and increase throughput. For certain nucleic acid sequencing applications, the method provides:

Highly multiplexed reactions and readout. Using a DNA gridding robot, it is straightforward to densely array 10,000 different DNA samples (or PCR derivatives) onto a single 2-D surface. Moreover, the method allows for a large multiplexing within each sample's PCR. Performing one sequencing reaction across an entire surface greatly reduces reagent costs and sequencing time.

Inexpensive machines and reagents. The method decouples several steps, including PCR amplification, robotic gridding, surface DNA synthesis, and fluorescent scanning. For each step, relatively inexpensive off-the-shelf equipment and protocols already exist. Appropriate selection of nonproprietary reagents can further reduce overall costs.

Reduced number of required experiments. For STR analysis and mutation detection applications, the desired information is far less than the amount available in the full DNA sequence. The method exploits this information reduction by requiring relatively few experiments.

More informative markers. SNPs are not ideal genetic markers; their attractiveness lies primarily in their scalability via DNA arrays. The new method confers the advantages of DNA arrays to more powerful genetic markers (STRs, sequences, and other polymorphisms). This novel scalability creates more options on which to build future genetic assay platforms.

This application introduces new methods relative to U.S. PTO application No. 09/301,917, entitled “A Method and System of DNA Sequencing,” filed by the inventor on Apr. 29, 1999, incorporated by reference in its entirety. One novel feature includes the use of DNA termination chemistry and Laplace transform analysis. Among other elements, the array substrates, separation-free detection mechanisms, and biological applications described in 09/301,917 are applicable to this invention, and are incorporated by reference.

### VI. DNA Transform Sequencing

A DNA sequence's information can viewed as four signals—one for each base. Each signal encodes the positions at which the base occurs in the sequence. By introducing a predetermined amount of base terminator into the sequencing reaction, a damping effect is achieved. Greater damping (i.e., more terminator) reduces the observed total signal.

The total signal can be measured as a 0-D result from a single tube, microtiter well, or array dot. Moreover, the damping reduction follows the mathematics of the Laplace Transform. Since the Laplace is an information preserving transform, DNA sequence information can be inferred from these measurements.

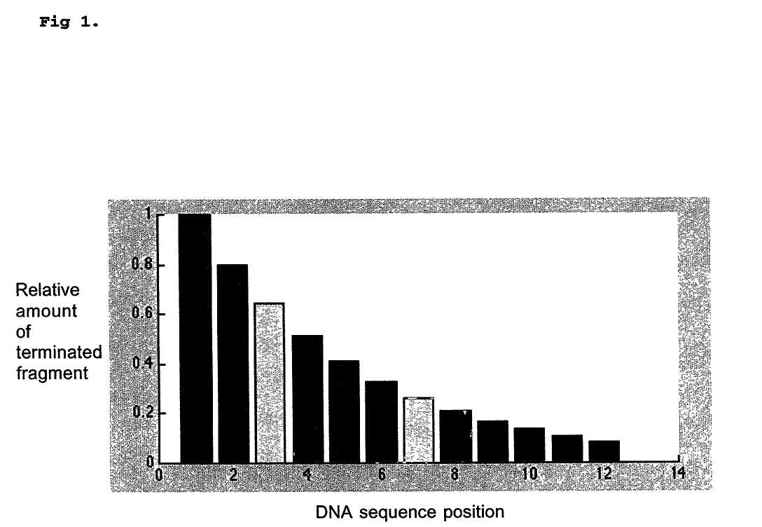

By applying an equal damping effect to all four bases, one can measure the Laplace transform coefficients of an arbitrary DNA sequence. Referring to FIG. 1, a damped DNA ladder is shown with the degree of damping set by the amount of terminator present. The Laplace coefficient for each base is the proportion of that base's label relative to all the bases.

A key use of this method is for analyzing DNA ladders using labeled ddNTP analogs and conventional dideoxy terminator chemistry in order to determine part or all of a DNA sequence. For clarity, however, the exposition starts with a simpler system —sizing one or two DNA fragments (rather than an entire sequencing ladder).

### VII. Fragment Sizing System

The system described herein can be readily adapted for use in any nucleic acid fragment sizing application. Such fragment sizing applications may include differential display of expressed genes, amplified fragment length polymorphism, single nucleotide polymorphism, short tandem repeats, gene dosage, and so on; these useful applications are detailed in the section below on “Fragment sizing applications”. For clarity of exposition, a detailed STR microsatellite example is presented here.

Consider the CA-repeat STR sequence (CA)ₙG. By adding ddATP terminator to the sequencing reaction, a spectrum of sequencing products results, reflecting the early termination of some fragments. Arranged by increasing length, these products are CA, CACA, CACACA, . . . , (CA)ₙG.

The relative amounts of each product depend on the probability p of extending the sequence at an A position. This probability can be written as the ratio of chemical concentrations:

$$p = \frac{[ dATP ]}{[ dATP ] + α [ ddATP ]}$$

where [X] denotes the concentration of species X, and α is the polymerase reaction dependent incorporation efficiency of the nucleotide terminator ddATP relative to the nucleotide DATP. Let q be the probability of termination at an A position, where q=1−p.

One preferred embodiment for calibrating the incorporation efficiency a entails using the preceding chemical equation for fitting data. For example, rewriting the chemical equation into a more convenient form, for each experiment i:

$$\frac{{p}_{0}}{{p}_{i}} = 1 + {α ( \frac{[ ddATP ]}{[ dATP ]} )}_{i}$$

where p₀ is the maximum observed signal corresponding to [ddATP]=0. Using a DNA template containing a single repeat, collect data for specific ratios of [ddATP] to [dATP], and record the peak signal Pᵢ, and observe the magnitude of detected label. Error minimization of the linear model then estimates the parameter α.

From the extension probability p, one can compute the probabilities of forming each fragment. These are:

| | |
|---|---|
| CA | q |
| CACA | pq |
| CACACA | p²q |
| (CA)ₙ | pⁿ⁻¹q |
| (CA)ₙG | pⁿ |

Since q(1+p+. . . +pⁿ⁻¹) equals (1−pⁿ), the sum of these probabilities is 1, so all events are accounted for.

Note that the probability of forming each fragment scales as an inverse exponential function of the length of the fragment. This damping effect is mathematically related to the kernel of the Laplace Transform. The precise relationship depends on how the fragments are labeled. Suppose there are labels only on the 3′-end G nucleotide. Then the detected signal of a CA-repeat with n repeats would be proportional to pⁿ.

In the preceding homozygote case of one allele, knowing pⁿ immediately gives the repeat size n. With heterozygotes, two data points are needed to determine the two unknowns. This can be done by solving a linear matrix equation. For the simple case of three size alleles (CA)₁, (CA)₂, and (CA)₃, this equation is written as:

$$[ \begin{matrix}{d}_{1} \\ {d}_{2} \\ 1\end{matrix} ] = [ \begin{matrix}{p}_{1} & {p}_{1}^{2} & {p}_{1}^{3} \\ {p}_{2} & {p}_{2}^{2} & {p}_{2}^{3} \\ \frac{1}{2} & \frac{1}{2} & \frac{1}{2}\end{matrix} ] \cdot [ \begin{matrix}{a}_{1} \\ {a}_{2} \\ {a}_{3}\end{matrix} ]$$

where aᵢ are the alleles (taking on integer values 0, 1 or 2), pᵢ are the extension probabilities used in the two experiments, and dᵢ are the observed data. The third row is the constraint that two alleles are present.

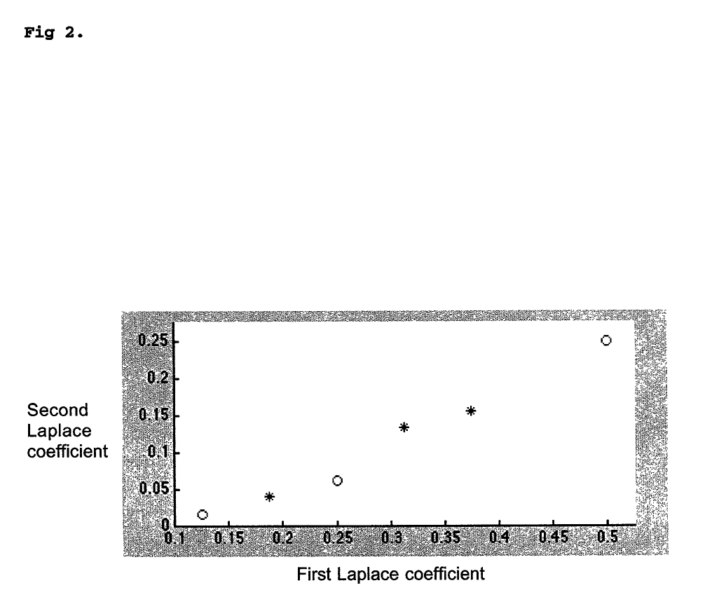

The three alleles (in a locus) case was addressed with the two experiments where p₁=0.50 and p₂=0.25, using numerical simulation in MATLAB (The MathWorks, Natick, Mass.). The six simulated [d₁ d₂] data pairs were generated for the six genotype cases (the heterozygotes [1 1 0], [1 0 1], [0 1 1], and the homozygotes [2 0 0], [0 2 0], [0 0 2]). These data pairs (each corresponding to a unique genotype) formed numerically distinct cluster regions, referring to FIG. 2. Directly solving the matrix equation using MATLAB's matrix inversion operation on the data recovered the exact genotype values.

This analysis shows that DNA fragment length genotypes can be determined without performing a 1-D DNA size separation. Instead, one can conduct two 0-D (tube or dot) experiments using two different ddATP to dATP terminator ratios. The resulting measurements are Laplace coefficients that contain enough information to mathematically estimate the fragment sizes.

The transform method can handle any number of alleles or fragment sizes. Additional experiments (at varying ddATP to dATP terminator ratios) enable transforms with more data and sizing points. Since the Laplace transform is quantitative, real-valued nonintegral DNA concentrations can be estimated at the different sizes from the data. This feature is useful in quantitative analysis of nucleic acid sizing assays, including processing STR artifacts, AFLP, differential display, DNA sequence ladder determination, SSCP, gene dosage, SNP measurements, and using pooled DNA templates from multiple individuals.

The method's general applicability to nucleic acid fragment sizing suggests a method of nucleic acid sequencing comprising the steps:

(a) amplifying a nucleic acid sample to produce an amplified DNA product;

(b) extending a sequencing primer bound to the DNA product in the presence of terminating nucleotide analogs to produce a collection of labeled nucleic acid products;

(c) detecting a total amount of label present in the collection to produce a measurement; and

(d) combining a plurality of measurements to determine DNA sequence information about the sample.

### VIII. Chemistry

In the method of nucleic acid sequencing, referring to step (a), amplifying a nucleic acid sample to produce an amplified DNA product:

An experiment was conducted that used synthesized CA-repeat oligonucleotide templates. The three templates contained (GT)ₙ, n=1, 2, 3, and were 5′ biotinylated for purification steps. The sequencing primer was fluorescently labeled (NED dye; PE Biosystems, Foster City, Calif.) on the 5′ end in order to estimate quantities related to the number of DNA strands. A poly-A tail was added for better sequencer detection. The complementary sequences used were:

5′-NED-A₁₀-GTTTTCCCAGTCACGA-3′

3′-CAAAAGGGTCAGTGCT-(GT)ₙ-CCAA-Biotin-5′

Extension from the sequencing primer forms a (CA)ₙ subsequence, followed by a G. The biotinylated “. . . GCT-(GT)ₙ-CCA . . . ” template shall be loosely referred to herein by its complementary “(CA)ₙG” name.

In the Sequenase (USB, Cleveland, Ohio) extension reaction, the nucleotide precursors used were:

dCTP,

dATP and ddATP (Amersham, Piscataway, N.J.), in predetermined ratios, and

ddGTP-JOE, labeled with the fluorescent JOE dye (NEN Life Science Products, Boston, Mass.).

The ddATP:dATP ratio was set to achieve a desired extension probability p. No TTP precursors were used. Thus, sequence termination could occur by either:

ddATP, which prematurely terminated the (CA)ₙG sequence, or

ddGTP, which labeled and terminated the full-length (CA)ₙG sequence.

The result of a sequencing reaction is a collection of 5′ labeled molecules (n=1,2,3):

5′-NED-A₁₀-GTTTTCCCAGTCACGA-(CA)ₙ-3′

along with a full-length molecule labeled at both the 5′ and 3′ ends:

5′-NED-A₁₀-GTTTTCCCAGTCACGA-(CA)₃-G-JOE-3′

The ratio of the observed total JOE to total NED fluorescent dye intensities is therefore a measure of the fraction of full-length molecules (relative to all the molecules). This fraction is a function of the extension probability p used in the mathematical analysis. And, the functional form relating the p that is set to the ratio we observe is precisely the Laplace transform, from which one can determine the DNA sizes.

### IX. Extension on Substrate

In the method of nucleic acid sequencing, referring to step (b), extending a sequencing primer bound to the DNA product in the presence of terminating nucleotide analogs to produce a collection of labeled nucleic acid products:

The sequence extension reactions were conducted in streptavidin-coated plates. This section describes the protocols used.

Immobilization. Reacti-Bind™ streptavidin-coated polystyrene strip plates (Pierce, Rockford, Ill.), were used, with Blocker™ BSA. The plates were washed 3× with 200 μL of TBS buffer (25 mM TRIS and 150 mM NaCl; pH=7.2) by shaking at room temperature. To immobilize the template, 3 μL Binding Buffer (5 mM EDTA, 5× Denhardt's and 0.1% Tween 20 in TBS) and 1 μL [1 μM] biotinylated sequencing template (1 pM) (Gibco BRL, Life Technologies, Rockville, Md.) were added. The solution was incubated at room temperature for 15 minutes, and then washed 3× (repipetting 3×) with 200 μL washing buffer (0.3% Tween 20 in TBS).

Extension. 2 μL [5×] of Sequenase reaction buffer (USB Corporation, Cleveland, Ohio) was combined with 1 μL [1 μM] (1 pM) of the NED-labeled sequencing primer. These were incubated at 65° C. for 6 min in a thermal cabinet (Biometra, OV/5), and then further incubated at 37° C. for 25 min. Additional reagents were then added, including:

1 μL [50 μM] (50 pM) dATP (Promega, Madison, Wis.)

2.5 μL [20 μM] (50 pM) dCTP (Promega, Madison, Wis.)

5 μL [10 μM] (50 pM) ddGTP-JOE (NEN Life Sci, Boston, Mass.)

1 μL [10 U/μL] Sequenase (USB Corporation, Cleveland, Ohio)

x μL [100 μM] ddATP (variable) (Amersham, Piscataway, N.J.)

deionized Water (variable), filling to 17.5 μL total volume

For sequencing extension, the reaction mixture was incubated at room temperature for 25 min. Washing was done 3× with 200 μL of washing buffer.

For improved enzyme stability, 1 ul of 0.1M dithiothreitol (DTT) can be added to a primer-template mix after the annealing step. This brings the final concentration of DTT in a 15 ul extension reaction to about 7 mM. It is useful to prepare a master mix containing DTT and dNTPs, and then add this to the primer-template mix after the annealing step, and then add 2 ul of T7 Sequenase (3.25U) to start the extension.

Denaturation. To remove the nonbiotinylated strand, 20 μL of deionized formamide was added, denaturating on a heatblock at 95° C. for 5 min. 2 μL of this sample was then added to 12 μL of deionized formamide prior to loading onto an ABI/310 automated DNA sequencer.

Extension without terminators. There are situations where the amount or quality of DNA template is a limiting factor. In an alternative preferred embodiment, PCR of one or more sites is done on such a template using a set of unlabeled PCR primers. The sequencing extension reaction in this embodiment does not use ddNTP terminators to generate Laplace transform data. The sequencing extension primer can be labeled, or, alternatively, the labeling can be done via incorporation or termination. The extension reaction synthesizes a full-length DNA product, since Laplace-inducing terminators are not used. The readout detection of said full-length product is done on a sequencing instrument. The lengths of the sequencing primers can be varied (e.g., using poly-A upstream headers, molecular weighting molecules, longer sequences of upstream DNA, etc.). The effect is that (a) PCR can amplify very short PCR regions, while (b) the electrophoretic readout can be multiplexed by the varying mobility of the extension products. This type of assay (short PCR regions, arbitrarily sized labeled readout fragments) has particular application when a DNA template is degraded or in a limiting quantity. Such situtations arise in forensics, human identity, and genetic studies.

### X. Detection

In the method of nucleic acid sequencing, referring to step c, detecting a total amount of label present in the collection to produce a measurement:

To best understand the sequencing extension products, these products were size separated the on an ABI/310 single capillary Genetic Analyzer (PE Biosystems, Foster City, Calif.). A 14 μL loading volume was used, with the POP4 gel, an STR capillary, and filter set F. The run time was 20 min, at a run temperature of 60° C. The peak heights and areas were estimated using PE's GeneScan software. Initial calculations were done in Microsoft Excel on an Apple Macintosh computer.

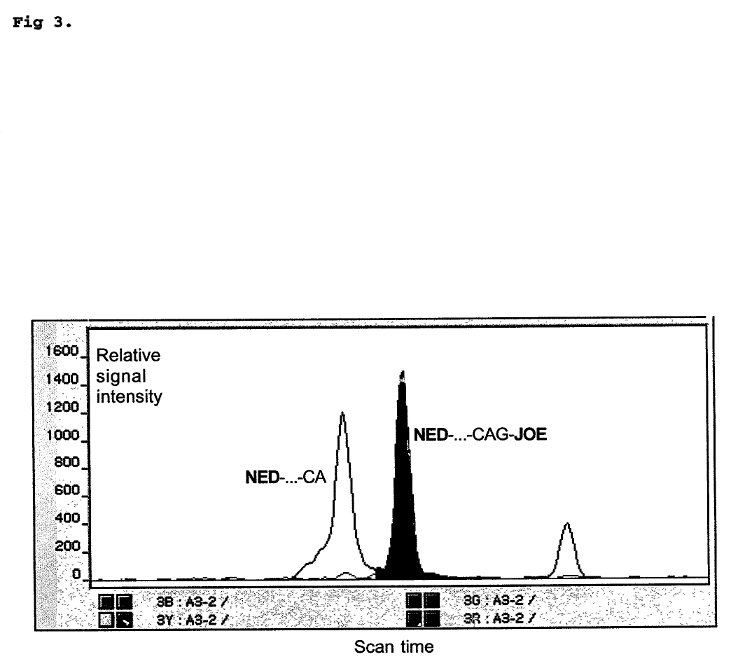

Using the (CA)₁G template, it was determined that a ddATP:dATP ratio of 2:1 (i.e., 100 pM ddATP and 50 pM DATP) roughly corresponded to an extension probability of 0.5. Referring to FIG. 3, this was done by checking for roughly equal heights (in the 5′ strand end NED dye) of the (CA)₁ ddATP

For the key experiments, 18 reactions were performed. Three (approximate) extension probabilities were used:

p=0.25 (300 pM ddATP),

0.50 (100 pM ddATP), and

0 0.75 (33 pM ddATP).

These experiments were done for all six possible genotypes (two alleles selected from three choices), using the template combinations:

1, 2, 3, 1+2, 1+3, 2+3

where “n” denotes the template for (CA)ₙG, and “m+n” denotes equimolar quantities of the (CA)ₘG and (CA)ₙG templates.

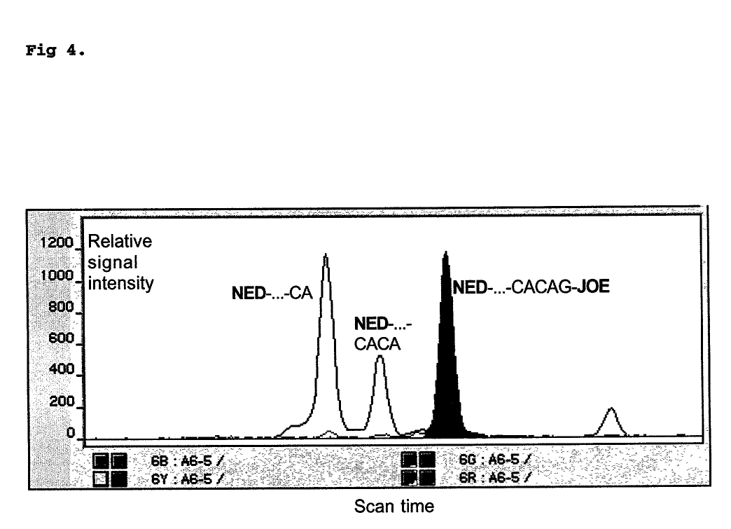

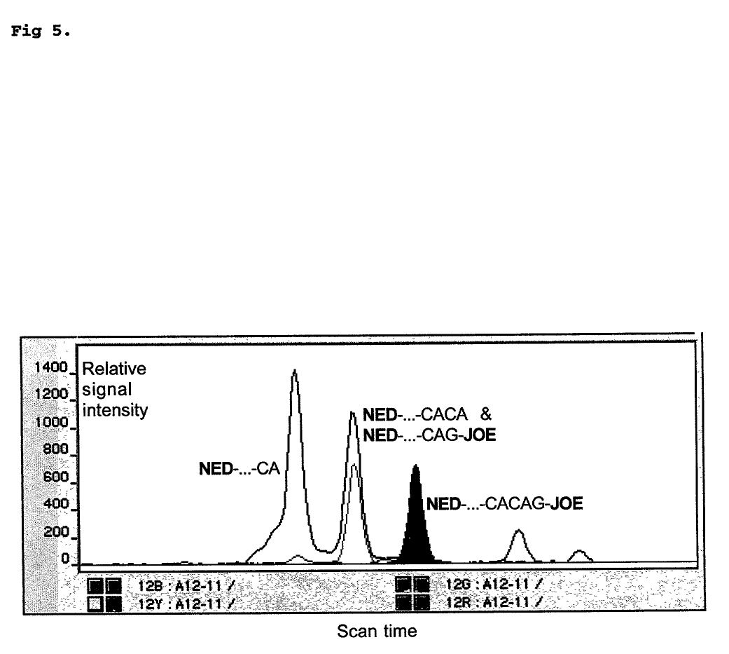

Referring to FIG. 4, the electrophoretograms are shown for a homozyotic genotype (template 2) experiment. Referring to FIG. 5, the electrophoretograms are shown for a heterozygotic genotype experiment (templates 1+2). The peak heights were tabulated for each dye from the GeneScan data, and used as estimates of DNA concentration.

### XI. Analysis of Transform Data

In the method of nucleic acid sequencing, referring to step d, combining a plurality of measurements to determine DNA sequence information about the sample:

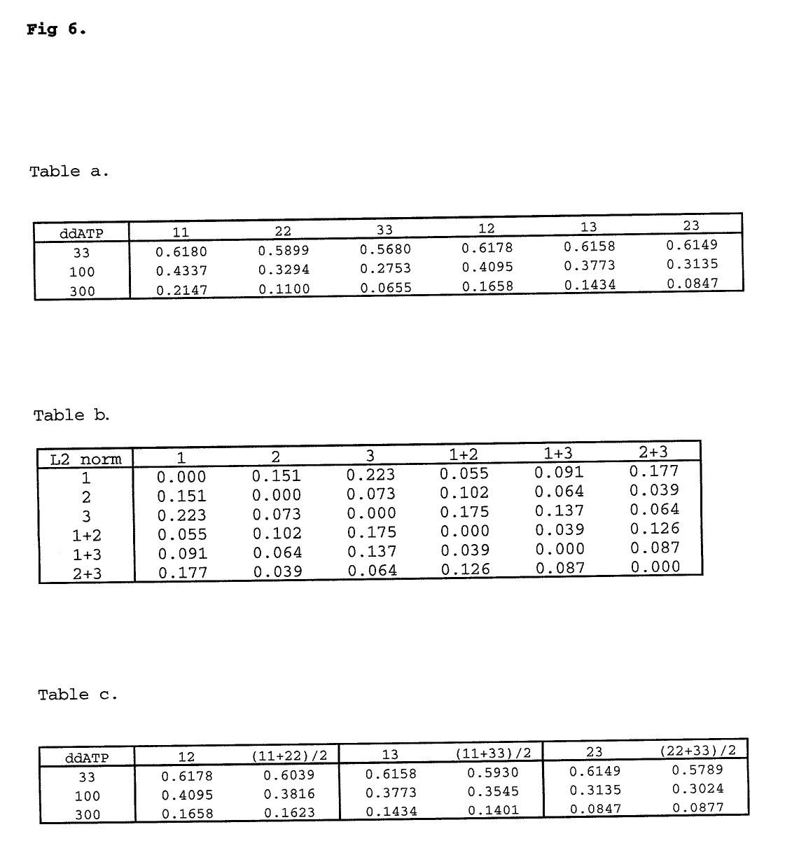

For each experiment, the ratio of the JOE (3′ terminator) signal to the NED (5′ strand) signal was computed from the fluorescent data. For a single DNA fragment, this ratio decreases exponentially with the fragment length. For two fragments, the ratio can be predicted by theory or calibrated from the data. For each STR genotype, these ratios recorded for different ddATP damping experiments can be used as a signature for calling the genotyping. Referring to FIG. 6, the signatures of the six genotypes in our pilot system are shown in Table a.

The cluster signatures are quite distinguishable from each other. To demonstrate this, the Euclidean distances between all signature pairs were computed. Referring to FIG. 6, the results are shown in Table b. These results show that the system can distinguish the signatures from one another, and robustly ascertain the genotypes.

A useful check on the data is examining how well they conform to the linear matrix model. For example, theory predicts (and observation confirms) that the heterozygotic genotype curve of FIG. 5 can be formed by adding together the curves of the homozygotic genotypes of FIGS. 3 and 4. This hypothesis can be tested by comparing each observed heterozygote signature with the average of the observed signatures of its homozygote components. Referring to FIG. 6, these comparisons are shown in Table c. The analysis is consistent with the underlying linear model.

Much information can be computed from such a data set. The relative efficiency α of ddATP incorporation was estimated in this case to be 0.41, relative to dATP. The extension probability p was computed for each ddATP amount used. Other model assumptions were checked against the data. This compability of data and model demonstrates the correctness and utility of the DNA transform sequencing approach.

### XII. Microtiter Plate Embodiment

The above results illustrated the method's operation in a one tube reaction. The DNA transform sequencing data were generated for DNA fragments, and their size then determined without electrophoresis. In an alternative preferred embodiment, DNA transform sequencing is conducted as a microtiter plate assay (e.g., in 96-well, 384-well, or larger formats). As described later in this specification, techniques used for the microtiter plate parallelization also apply to highly parallelizable surface assays (such as DNA microarrays).

### Chemistry

In the method of nucleic acid sequencing, referring to step (a), amplifying a nucleic acid sample to produce an amplified DNA product:

Polymerase. The preferred embodiment uses Sequenase (modified T7), a highly processive DNA polymerase without 3′ exonuclease activity that readily incorporates nucleotide precursor analogs such as ddNTPs and labeled bases (Tabor, S., and Richardson, C., 1987, “DNA sequence analysis with a modified bacteriophage T7 DNA polymerase,” Proc Natl Acad Sci USA, 84(14): 4767-71), incorporated by reference. These properties work well in DNA transform sequencing, and help implement the underlying mathematical requirements. In an alternative preferred embodiment, nonproprietary polymerase enzymes can be sued, such as the Klenow fragment. These enzymes have utility for short sequencing runs, and can reduce the cost of the reactions.

Labels. The most preferred embodiment used two fluorescent dyes. In an alternative preferred embodiment, this number can be increased to 3, 4 or 5 dyes. The simultaneous use of more labels can provide information about more than one sequencing ladder at a time, thereby reducing the time and cost of the method.

Template. The described embodiment used long, synthesized oligonucleotides as the nucleic acid template. The most preferred preferred embodiment uses PCR products as sequencing templates. These products are formed from a forward primer, and a biotinylated reverse primer. Following denaturation, the sequencing reaction is then primed on the biotinylated reverse DNA strand. Moreover, this amplification can be done in a multi-well (e.g., 96 or 384) format using a PCR thermocycler (PTC-100; MJ Research, Watertown, Mass.) that can amplify in a multi-well plate format.

Primers. In the most preferred preferred embodiment, multiple PCR primer pairs are combined into a single multiplex PCR, and then reliably measured. Ordinary fluorescent detection of size separated DNA has limited multiplexing power, due to the requirement that all signals simultaneously appear within a narrow common detection range on the readout lane of the gel or capillary. However, DNA transform sequencing does not have this limitation. By counting (and normalizing by) the number of sequencing strands (e.g., using a 5′ label on the sequencing primer), and performing a separate sequence detection for each PCR product, one can quantitatively detect fluorescence over a much wider dynamic range. This flexibility greatly increases PCR multiplexing.

Nucleotides. A variety of different fluorescently labeled ddNTP analogs can be used. These analogs enable several desirable assay properties:

Eliminate the 5′ primer label. Currently, the 5′ label is used to normalize the signals. However, exploiting the transform mathematics, one can normalize the signals by mixing in other ddNTP 3′ terminator labels, in place of the 5′ label. This simplification can reduce the eventual cost of the assay, since no dye-labeled oligo is then required in the assay. This effect reduces oligo costs, and eliminates the need to attach proprietary dyes.

General DNA sequencing. Using multiple detectable terminators helps design robust DNA sequencing assays. This is further described in the next section.

Higher throughput. Simultaneous readout from multiple bases increases the throughput of the sequencing assay.

### Substrate

In the method of nucleic acid sequencing, referring to step (b), extending a sequencing primer bound to the DNA product in the presence of terminating nucleotide analogs to produce a collection of labeled nucleic acid products:

The protocols above can be performed manually in strip tubes using hand pipettors. For more parallelization and better reproducibility, an automated parallel format (e.g., 96-well) is preferred. One preferred embodiment uses 96-well streptavidin-coated microtiter plates (regular or thin-wall) as the DNA solid support; these plates are commercially available from several suppliers (e.g., Xenopore, Hawthorne, N.J.). Pipetting is done using a 96-channel Hamilton syringe semi-automated robot, such as the Hydra-96 device (Robbins Scientific, Sunnyvale, Calif.), and washings done using an automated plate washer (e.g., ELx405 from Bio-Tek, Winooski, Vt.). The single tube protocols immediately apply to the parallel and scalable DNA support formats.

### Detection

In the method of nucleic acid sequencing, referring to step c, detecting a total amount of label present in the collection to produce a measurement:

The embodiment described used an ABI/310 capillary electrophoresis system for size separating and fluorescently detecting the DNA fragments. While this approach is well-suited to protocol development and troubleshooting, a key rationale for DNA transform sequencing is eliminating entirely such gel electrophoresis instruments from the sequence analysis process. For microtiter plate applications, the most preferred embodiment uses a multi-well microplate fluorescence reader to measure the signals in the detection assay. Such readers (e.g., 96-well) are available from several manufacturers (Beckman, Bio-Tek, Packard, etc.)

### Analysis

In the method of nucleic acid sequencing, referring to step d, combining a plurality of measurements to determine DNA sequence information about the sample:

Methodology. There are two most preferred embodiments for assigning data signatures to their appropriate sequence or genotype: clustering and modeling.

The clustering embodiment has the advantage of robustness—regardless of the underlying model, calibration data can be used to establish cluster points and assignment criteria.

The modeling embodiment has the advantage that with linear matrix mathematics, new innovations can be developed to exploit assay extensions and their associated linear algebra.

In their appropriate context, each method is a suitable embodiment for assay analysis.

Applications. Many applications, including some for genetic variation, are based on measuring multiple DNA fragment lengths. Other applications, such as mutation detection, require characterization of DNA sequence content. In both cases, it is useful to model the distributions (of fragments or sequencing ladders) as functions with assayable Laplace transforms.

Controls. It is useful to incorporate proper controls directly into the experiment. In one preferred embodiment, simple, known fragment lengths or sequences should be included in order to calibrate parameters or cluster points. Such calibration controls were used in the described fragment analysis situation, where the use of single fragment data helped predict the behavior of (potentially unknown) heterozygotic fragments. In the most preferred embodiment, known controls for simple function (and transform) behavior are included as assay point. These basis functions facilitate better analysis of more complex unknown sample behavior.

Sampling. From Laplace transform theory, one data point might suffice to distinguish two DNA sequences, and two data points should be enough determine two fragment lengths. However, when considering experimental error and the robustness of the result, more data transform samples may be helpful. In the two fragment data developed above, three (not just two) different ddATP ratios were used to help resolve the genotypes. In a most preferred embodiment, additional data samples are gathered in order to overdetermine the solution, and thereby robustly analyze the DNA signals in the presence of experimental noise, error, or uncertainty.

### XIII. Applications of the Transform Method

### Sizing

The DNA transform sequencing method can size STR PCR products. Consider the STR tetranucleotide repeat marker THO1, which is used in both genetic and forensic science. THO1's repetitive element is “TCAT”, so the described CA-repeat sizing protocol (with the inclusion of an unlabeled ddTTP) applies. Moreover, the PCR is quite robust (having several published PCR primer pairs), and the DNA sequence is well known.

The method is generally applicable to any tandem repeat sizing assay. For a locus of the form PQRₙST, P is the forward primer, Q the left flanking region, R is the repeat unit (repeated n times), S is the right flanking region, and T describes the reverse PCR primer. The sequencing primer is located in the PQRₙ region. Any number of alleles (e.g., including more than two) can be present, in arbitrary relative concentrations, since the Laplace transform operates over any finite vector in the real and complex fields. Although the single individual STR genotyping situation (where there are one or two integer values) is an important application, there are others. For example, pooling individual DNAs (pre- or post-PCR) finds application in many genetic applications, such as linkage disequilibrium studies.

Note that a 3′ terminator need not be used in the assay. In one preferred embodiment, the label (whose Laplace terminator decay helps determine fragment length) can be incorporated into the nascent DNA strand, rather than being present as a terminator. There is a minor adjustment to the formulas, but the essential decay property is retained in the detected data, which enables the Laplace transform mechanism to operate. When incorporating labeled nucleotides, it is useful to dilute the labeled dNTPs with unlabeled dNTPs, so as to reduce steric hindrance.

PCR artifacts from tandem repeat products are readily addressed using the method. Earlier work mathematically modeled (and eliminated) PCR stutter and relative amplification (Ng, S.-K., 1998, “Automating computational molecular genetics: solving the microsatellite genotyping problem,” Doctoral dissertation, CMU-CS-98-105, Carnegie Mellon University; Perlin, M. W., Burks, M. B., Hoop, R. C., and Hoffman, E. P., 1994, “Toward fully automated genotyping: allele assignment, pedigree construction, phase determination, and recombination detection in Duchenne muscular dystrophy,” Am. J. Hum. Genet., 55(4): 777-787; Perlin, M. W., Lancia, G., and Ng, S.-K., 1995, “Toward fully automated genotyping: genotyping microsatellite markers by deconvolution,” Am. J. Hum. Genet., 57(5): 1199-1210; Martens, H. and T. Naes, 1992, Multivariate Calibration, New York: John Wiley & Sons), incorporated by reference. The Laplace analysis methods are not restricted to binary or integer valued functions—they work on any real (or even complex) valued function. Therefore, calibration (as described in the literature) of stutter or other PCR artifacts (e.g., relative amplification) permits prediction and correction in quantitatively accurate data.

In one embodiment, these calibrations of reproducible PCR artifacts are performed prior to the DNA transform sequencing. In the most preferred embodiment, known control samples are used to calibrate the PCR artifacts, and the analysis phase uses these calibrations to automatically remove the artifacts from the data, and thereby more accurately score the data. With clustering algorithms, the correction adjusts to the new position of the clustering. With linear models, the correction transforms the linear space to new coordinates using the observed positions of the artifact-containing data.

### Sequencing

Fragment sizing for STR genotyping of single individual focues on finding the position of two fragments. DNA sequencing can be more complex: information is needed from all the fragments that lie on the base's sequencing ladder. However, the fundamentals of the DNA transform method are the same: perform experiments that provide Laplace transform coefficients, and then combine these numerical coefficients to derive useful sequence information.

Synchronized termination. To obtain the Laplace transform of a DNA sequence, it is preferable to have a uniform decay rate damping the base signals. This is done by choosing an extension probability p, and then setting each of the four ddNTP:dNTP ratios (N=A, C, G, T) to achieve p. (This ratio calibration was described above.) Then, to observe the A ladder (for example), sequence using a 5′ end-labeled sequencing primer, labeled ddATP (a different label), all other ddNTPs unlabeled, and the correct proportions of dNTPs. This reaction will form doubly labeled (5′ and 3′) molecules at positions where there is an A in the DNA sequence, and singly labeled (5′ only) molecules at the other positions. The ratio of the 3′ label to the 5′ label is then proportional to the Laplace coefficient at that decay probability.

Multiplexing. It is useful to obtain the Laplace coefficients of all four bases simultaneously in a single transform sequencing reaction. This can be done by using labeled ddNTPs for all four bases, with a different label for each ddNTP. (The ddNTP:dNTP ratios that achieve p using these labeled ddNTP precursors are recalibrated.)

One preferred embodiment for four base multiplexing is to use five different fluorescent dyes: one for each of the four ddNTPs, plus one more for the 5′ strand label. However, this embodiment has two negative features: (1) five color instruments are not yet generally available, and (2) there is an additional cost in using oligos that are 5′ labeled with (possibly proprietary) fluorescent dyes.

In the most preferred embodiment for four base multiplexed DNA transform sequencing, four dyes are used. The mathematics imposes a useful constraint—the sum of the four (appropriately calibrated) ddNTP components equals unity. Therefore, the 5′ strand label is not strictly necessary for normalization, since the observed sum of the four dye intensities can be used for normalization instead.

From a chemistry perspective, this four base DNA transform sequencing embodiment is essentially equivalent to a standard four dye terminator Sanger-style sequencing reaction. The key differences are that:

precisely calibrated amounts of labeled ddNTP:dNTP ratios are used;

with much larger quantities of ddNTP; and

there is no size separation—

instead, detection is performed on the entire unseparated labeled product.

This nonobvious use of off-the-shelf sequencing chemistry is useful for enabling technological and commercial success.

Partial information. With an unknown DNA sequence, transform theory suggests that n experiments are needed to decipher a sequence n bases long. This experiment-intensive approach can be useful in some limited situations, such as large-scale population sequencing on high-density microarrays. However, for the more common clinical situation of mutation detection, there is much information known in advance, and this information greatly reduces the experimentation requirements.

With m known gene mutations, the task can be viewed as distinguishing between these mutations, and selecting the correct one. A single quantitative observation might (in principle) distinguish m cases. However, log₂(m) experiments is a more typical data requirement. For example, to robustly distinguish 4 possible mutations, only 2 experiments are needed. In an array format, each experiment might be conducted on tens of thousands of samples simultaneously. This potential for a vast reduction in the number of required experiments is a highly useful feature of DNA transform sequencing for detecting mutations in well-characterized genes.

### Cancer

Fragment analysis. DNA transform sequencing can perform low-cost scalable fragment analysis experiments on tumor materials. Specifically, each standard cancer genetics STR assays (e.g., STR genetic markers, microsatellite instability, and loss of heterozygosity) can be implemented in a DNA transform version.

Sequence analysis. DNA transform sequencing experiments can be performed on tumor material for detecting mutations, where several bases have changed in a small gene region. Note that:

This multi-base change situation is not amenable to SNP minisequencing.

A full 500 bp sequence read is quite costly relative to the information obtained.

Focused DNA chip technology is intolerant of new mutations, with high set-up costs.

The scalable DNA transform sequencing method greatly reducs the cost-per-bit in such cancer-related sequence analysis.

### XIV. Array Format Experiments

Arrays. The most preferred embodiment uses array surfaces, instead of 96-well microtiter arrays. This format reduces the cost of the sequence extension reaction by distributing small reagent volumes over very many DNA samples. DNA arrays also compress the samples into a small area, which enables a high-density readout. When the PCR products are deposited on a surface (or located in a tube or microtiter well), the probing mixture includes a specific sequencing primer, along with ddNTP and DNTP precursors in appropriate ratios. These primers and precursors can be multiplexed for greater efficiency.

Macroarray format. A conventional robotic macroarraying device (e.g, BioGrid, BioRobotics, Malden, Mass.) deposits 1,000 to 100,000 PCR-amplified samples onto a surface (e.g., 8×12 cm nylon membrane) suitable for hybridization, extension, washing, and readout. The specific sequencing primer extension in the presence of fluorescently labeled dNTPs and terminating analogs is performed on this surface. This extension is preferrably performed using a hybridization incubator optimized for the surface media, such as a standard hybridization oven. After washing, the quantitative detection of the fluorescent signal is done on a flat-bed laser scanner, such as the Hitachi FM/BIOII. The high-density gridded data is automatically scored using array reading software.

Microarray format. A modern robotic microarraying device (Omnigrid, GeneMachines, San Carlos, Calif.; MicroGrid II, BioRobotics, Malden, Mass.) deposits 1,000 to 100,000 PCR-amplified samples onto a surface (e.g., glass microscope slide, or silicon surface) suitable for hybridization, extension, washing, and readout. The PCR products bind to the surface using an attachment chemistry, such as coating the surfact with lysine or streptavidin; with streptavidin, one PCR primer is biotinylated. The DNA transform sequencing primer extension is done in the presence of fluorescently labeled dNTPs and terminating NTP analogs directly on this surface. This extension is preferrably done using a hybridization incubator optimized for the surface medium (GeneMachines HybChamber, San Carlos, Calif.; Molecular Dynamics, Sunnyvale, Calif.). After washing, quantitative detection of the fluorescent signal is performed on a microarray laser scanning detector, such as the GSI Lumonics ScanArray 5000 (GSI, Kanata, ON) or the GenePix 4000A (Axon, Foster City, Calif.). The high-density gridded data is automatically scored using array reading software, such as QuantArray or GenePix Pro.

Immobilized materials. The above “Format I” approaches have the PCR products immobilized onto a solid support (e.g., glass slides, nylon membranes, streptavidin-coated tubes or microtiter plates) using robotic deposition. The invention then exposes these PCR products to a set of sequencing oligonucleotides either separately or in a mixture. This PCR product immobilization attachment approach is often referred to as a “DNA microarray” (R. Ekins and F. W. Chu, “Microarrays: their origins and applications,” Trends in Biotechnology, 1999, 17, 217-218), incorporated by reference.

Format II. Next described are the “Format II” approaches, where an array of sequencing oligonucleotides (e.g., 20 to 25-mers) or peptide nucleic acid (PNA) probes are synthesized either in situ (on-chip), or by conventional synthesis followed by on-chip immobilization. The oligo array is exposed to PCR products of the sample DNA, hybridized, and then extended using appropriate labeled DNTP and ddNTP ratios. Fluorescent detection quantitatively measures the amount of label present. Such arrays are related to the Affymetrix “DNA chip” or “GeneChip®” technology. Traditionally, DNA oligo chips are limited to simple hybridization or single base termination extension. However, the described invention uniquely includes a multibase DNA sequencing extension step. Moreover, the invention's multiple experiments are distinguished over the prior art in that they determine Laplace Transform coefficients which are used to reconstruct information about DNA sequence length or composition.

In an alternative “Format II” preferred embodiment, the specific sequencing oligos are bound to a solid support. Each sequencing oligo is a nested primer specific to the amplified locus, gene or other chromosomal region, and is the initiation point for DNA transform sequencing. The amplified sample PCR products are then placed in contact with the oligo surface, in the presence of a predetermined ratio of DNTP and ddNTPs (some of which are fluorescently labeled), along with the necessary sequencing enyzme, buffer, and other reaction elements. A plurality of experiments corresponding to different predetermined NTP ratios are performed to interrogate one chromosomal region. The amplified sample preferrably contains PCR products from multiple chromosomal regions. Multiple experiments are performed for these different chromosomal regions and predetermined NTP ratios, each with its own readout step (up to the fluorescent multiplexing capability of the readout instrument).

The DNA transform sequencing extension is preferrably done using a hybridization incubator optimized for the surface medium (GeneMachines HybChamber, San Carlos, Calif.; Molecular Dynamics, Sunnyvale, Calif.). After washing, quantitative detection of the fluorescent signal is performed on a microarray laser scanning detector, such as the GSI Lumonics ScanArray 5000 (GSI, Kanata, ON) or the GenePix 4000A (Axon, Foster City, Calif.). The high-density gridded data is automatically scored using array reading software, such as QuantArray or GenePix Pro.

Throughput example. DNA transform sequencing permits greater PCR multiplexing. Single-tube multiplexes of 10-15 STR markers are routinely done (e.g., as in forensic identification); since the invention eliminates some dynamic range limitations, a 25-plex PCR is feasible. Therefore, (25 markers)×(10,000 samples) yields 250,000 reactions per run. Performing 4 runs per day would amount to 1,000,000 “bits” per day. The use of very small volumes and nonproprietary reagents would further reduce substantially the per-reaction costs. The invention can achieve a 1¢ or less “cost-per-bit,” which is a 100-fold cost reduction relative to current methods.

Utility note. At 1ç per bit, the cost of a complete, highly-informative 10,000 STR marker genome screen for one individual would be $100. The scalable DNA transform sequencing assay thus enables many medically useful population-wide screens (for cancer monitoring, gene mutations, etc.). When coupled with phenotypic information, such affordable dense genetic profiling enables practical prospective medicine. The ability to accurately predict genetic risk will have a profound effect on society's ability to customize medicine to the individual patient, and thereby far more effectively prevent cancer and other diseases.

Multiple priming sites. The Laplace transform can have a limited effective range, particularly in the presence of noisy data. The DNA transform invention overcomes this limitation by performing additional experiments. One embodiment, described above, performs redundant experiments to overdetermine the solution; similarly, repeating experiments can reduce experimental error. The most preferred embodiment uses multiple sequence priming sites, preferrably spaced every 5-10 bp downstream from the initial priming site. Each such offset priming experiment (repeated using appropriate dyes and NTP ratios) provides focused information for a 2-20 bp region. Combining the analyzed results of these offset experiments provides more extensive information about the length or content of the DNA sequence fragment.

Alternative labels. While fluorescence provides convenient labeling for the DNA transform sequencing assay, any alternative labeling embodiments that provide for quantitative detection of the NTPs and their ratios are usable in the labeling and detection steps of the invention. Radioactive labels can be used, with double labeling done using two different isotopes,such as ³³P and ³⁵S. Any detectable nonradioactive label can be used (Kricka, L. J., ed. Nonisotopic Probing, Blotting, and Sequencing, Second ed. 1995, Academic Press: San Diego, Calif.), incorporated by reference. It is useful for the detection assay to provide a quantiative measurement of DNA concentration.

### XV. Fragment Sizing Applications

Genotyping data can be used to determine how mapped markers are shared between related individuals. By correlating this sharing information with phenotypic traits, it is possible to localize a gene associated with that inherited trait. This approach is widely used in genetic linkage and association studies (J Ott, Analysis of Human Genetic Linkage, Revised Edition. Baltimore, Md.: The Johns Hopkins University Press, 1991; N Risch, “Genetic Linkage and Complex Diseases, With Special Reference to Psychiatric Disorders,” Genet. Epidemiol., vol. 7, pp. 3-16, 1990; N Risch and K Merikangas, “The future of genetic studies of complex human diseases,” Science, vol. 273, pp. 1516-1517, 1996), incorporated by reference.

Genotyping data can also be used to identify individuals. For example, in forensic science, DNA evidence can connect a suspect to the scene of a crime. DNA databases can provide a repository of such relational information (CP Kimpton, P Gill, A Walton, A Urquhart, E S Millican, and M Adams, “Automated DNA profiling employing multiplex amplification of short tandem repeat loci,” PCR Meth. Appl., vol. 3, pp. 13-22, 1993; J E McEwen, “Forensic DNA data banking by state crime laboratories,” Am. J. Hum. Genet., vol. 56, pp. 1487-1492, 1995; K Inman and N Rudin, An Introduction to Forensic DNA Analysis. Boca Raton, Fla.: CRC Press, 1997; C J Fregeau and R M Fourney, “DNA typing with fluorescently tagged short tandem repeats: a sensitive and accurate approach to human identification,” Biotechniques, vol. 15, no. 1, pp. 100-119, 1993), incorporated by reference.

Linked genetic markers can help predict the risk of disease. In monitoring cancer, STRs are used to assess microsatellite instability (MI) and loss of heterozygosity (LOH)—chromosomal alterations that reflect tumor progression. (ID Young, Introduction to Risk Calculation in Genetic Counselling. Oxford: Oxford University Press, 1991; L Cawkwell, L Ding, F A Lewis, I Martin, M F Dixon, and P Quirke, “Microsatellite instability in colorectal cancer: improved assessment using fluorescent polymerase chain reaction,” Gastroenterology, vol. 109, pp. 465-471, 1995; F Canzian, A Salovaara, P Kristo, R B Chadwick, L A Aaltonen, and A de la Chapelle, “Semiautomated assessment of loss of heterozygosity and replication error in tumors,” Cancer Research, vol. 56, pp. 3331-3337, 1996;S Thibodeau, G Bren, and D Schaid, “Microsatellite instability in cancer of the proximal colon,” Science, vol. 260, no. 5109, pp. 816-819, 1993), incorporated by reference.

For crop and animal improvement, genetic mapping is a very powerful tool. Genotyping can help identify useful traits of nutritional or economic importance. (H J Vilkki, D J de Koning, K Elo, R Velmala, and A Maki-Tanila, “Multiple marker mapping of quantitative trait loci of Finnish dairy cattle by regression,” J. Dairy Sci., vol. 80, no. 1, pp. 198-204, 1997; S M Kappes, J W Keele, R T Stone, R A McGraw, T S Sonstegard, T P Smith, N L Lopez-Corrales, and C W Beattie, “A second-generation linkage map of the bovine genome,” Genome Res., vol. 7, no. 3, pp. 235-249, 1997; M Georges, D Nielson, M Mackinnon, A Mishra, R Okimoto, A T Pasquino, L S Sargeant, A Sorensen, M R Steele, and X Zhao, “Mapping quantitative trait loci controlling milk production in dairy cattle by exploiting progeny testing,” Genetics, vol. 139, no. 2, pp. 907-920, 1995; G A Rohrer, Li Alexander, Z Hu, T P Smith, J W Keele, and C W Beattie, “A comprehensive map of the porcine genome,” Genome Res., vol. 6, no. 5, pp. 371-391, 1996; J Hillel, “Map-based quantitative trait locus identification,” Poult. Sci., vol. 76, no. 8, pp. 1115-1120, 1997; H H Cheng, “Mapping the chicken genome,” Poult. Sci., vol. 76, no. 8, pp. 1101-1107, 1997), incorporated by reference.

Fragment analysis finds application in other genetic methods. Often fragment sizes are used to multiplex many experiments into one shared readout pathway, where size (or size range) serves an index into post-readout demultiplexing. For example, multiple genotypes are typically pooled into a single lane for more efficient readout. Quantifying information can help determine the relative amounts of nucleic acid products present in tissues. (G R Taylor, J S Noble, and R F Mueller, “Automated analysis of multiplex microsatellites,” J. Med. Genet., vol. 31, pp. 937-943, 1994; L S Schwartz, J Tarleton, B Popovich, W K Seltzer, and E P Hoffman, “Fluorescent multiplex linkage analysis and carrier detection for Duchenne/Becker muscular dystrophy,” Am. J. Hum. Genet., vol. 51, pp. 721-729, 1992; C P Kimpton, P Gill, A Walton, A Urquhart, E S Millican, and M Adams, “Automated DNA profiling employing multiplex amplification of short tandem repeat loci,” PCR Meth. Appl., vol. 3, pp. 13-22, 1993), incorporated by reference.

Differential display is a gene expression assay. It performs a reverse transcriptase PCR (RT-PCR) to capture the state of expressed mRNA molecules into a more robust DNA form. These DNAs are then size separated, and the size bins provide an index into particular molecules. Variation at a size bin between two tissue assays is interpreted as a concommitant variation in the underlying mRNA gene expression profile. A peak quantification at a bin estimates the underlying mRNA concentration. Comparison of the quantitation of two different samples at the same bin provides a measure of relative up- or down-regulation of gene expression. (S W Jones, D Cai, O S Weislow, and B Esmaeli-Azad, “Generation of multiple mRNA fingerprints using fluorescence-based differential display and an automated DNA sequencer,” BioTechniques, vol. 22, no. 3, pp. 536-543, 1997; P Liang and A Pardee, “Differential display of eukaryotic messenger RNA by means of the polymerase chain reactions,” Science, vol. 257, pp. 967-971, 1992; K R Luehrsen, L L Marr, E van der Knaap, and S Cumberledge, “Analysis of differential display RT-PCR products using fluorescent primers and Genescan software,” BioTechniques, vol. 22, no. 1, pp. 168-174, 1997), incorporated by reference.

Single stranded conformer polymorphism (SSCP) is a method for detecting different mutations in a gene. Single base pair changes can markedly affect fragment mobility of the conformer, and these mobility changes can be detected in a size separation assay. SSCP is of particular use in identifying and diagnosing genetic mutations (M Orita, H Iwahana, H Kanazawa, K Hayashi, and T Sekiya, “Detection of polymorphisms of human DNA by gel electrophoresis as single-strand conformation polymorphisms,” Proc Natl Acad Sci USA, vol. 86, pp. 2766-2770, 1989), incorporated by reference.

The AFLP technique provides a very powerful DNA fingerprinting technique for DNAs of any origin or complexity. AFLP is based on the selective PCR amplification of restriction fragments from a total digest of genomic DNA. The technique involves three steps: (i) restriction of the DNA and ligation of oligonucleotide adapters, (ii) selective amplification of sets of restriction fragments, and (iii) gel analysis of the amplified fragments. PCR amplification of restriction fragments is achieved by using the adapter and restriction site sequence as target sites for primer annealing. The selective amplification is achieved by the use of primers that extend into the restriction fragments, amplifying only those fragments in which the primer extensions match the nucleotides flanking the restriction sites. Using this method, sets of restriction fragments may be visualized by PCR without knowledge of nucleotide sequence. The method allows the specific co-amplification of high numbers of restriction fragments. The number of fragments that can be analyzed simultaneously, however, is dependent on the resolution of the detection system. Typically 50-100 restriction fragments are amplified and detected on denaturing polyacrylamide gels. (P Vos, R Hogers, M Bleeker, M Reijans, T van de Lee, M Hornes, A Frijters, J Pot, J Peleman, M Kuiper, and M Zabeau, “AFLP: a new technique for DNA fingerprinting,” Nucleic Acids Res, vol. 23, no. 21, pp. 4407-14, 1995), incorporated by reference.

### XVI. Other Applications

### DNA Sequencing

In modern molecular biology, genetics, and medical practice is often useful to determine the sequence of a DNA molecule. When there is some prior knowledge of the DNA sequence, as with resequencing or tandem repeat applications, the Laplace transform method is useful. The claimed invention can be used to replace Sanger (and related) DNA sequencing methods in currently performed sequencing applications, but with the potential advantages of higher parallelism, reduced experiment effort, greater speed, less tedium, and lower cost.

With the advent of whole-genome sequencing of human and other species, the invention can be combined with prior sequence data to devise powerful genetic assays. The sequence data provides information about STR, SNP, mutation, and other polymorphic sequences. The Laplace transform invention is used to elicit genetic variation information at these polymorphic genome regions from individuals or populations. Such human sequence data is now available (Venter, J. C., et al, The sequence of the human genome, Science, Feb. 16, 2001;291(5507):1304-51; Lander, E. S., Initial sequencing and analysis of the human genome, Nature. Feb. 15 2001;409(6822):860-921), incorporated by reference.

### Mutation Detection

For medical and gene discovery applications it is useful to detect chromosomal mutations by determining all or part of a DNA sequence. Mutations can be distinguished by determining the entire DNA sequence using the transform-based DNA sequencing methods specified herein. Other approaches, such as single-strand conformational polymorphism (SSCP), distinguish the mutations from each other by forming a representative signature for each mutation, but do not explicity determine every base in the DNA sequence. The transform-based DNA sequencing method specified herein is ideally suited to such partial signature approaches, since typically fewer experiments (e.g., in a mathematical transform space) are needed to distinguish many possible mutations. This information reduction translates into a tremendous reduction in the number of required experiments.

### DNA Diagnostics

An important class of mutations is DNA-based diagnosis for predisposition to genetic disease. For high-throughput screening, the most preferred embodiment of the transform-based DNA sequencing methods specified herein would deposit the amplified DNA at a genome locus of individuals as spots onto multiple copies of a two dimensional surface, with each spot corresponding to an individual. Transform-based sequencing would then obtain the partial sequence information about the m mutations that distinguish these mutations, without requiring a determination of the entire sequence. Since one hundred to a hundred thousand spots (i.e., different individuals) can be placed onto one surface for parallel experimentation, the time and cost of high-throughput DNA diagnostics is greatly reduced even further.

### Genetic Variation

It is often useful to study genetic variation in a population. Such variation has application in determining associations between populations and pharmacological effectiveness or side effects, discovering gene locations of inherited disease, and elucidating evolutionary pathways. The parallel detection feature of the transform-based sequencing method specified herein is ideally suited for all these applications. By partially characterizing the alleles of polymorphic loci of many individuals at high-throughput, large populations can be studied for low cost, effort, and time. One preferred embodiment of the invention for this application is the Laplace transform for genotyping tandem repeat length polymorphisms. Another preferred embodiment studies SNPs or other polymorphisms in the genome for a population.

### Forensics and Identification

In forensic science, a small set (e.g., 5-20) of highly polymorphic genetic markers are used to form a genetic fingerprint of an individual. These fingerprints can be compared to (a) match a stain with an individual or database (e.g., to convict a criminal), (b) genetically associate an individual with his relatives (e.g., paternity testing), and (c) identify an individual (e.g., deceased soldiers). Forensic fingerprinting has been described (A. J. Jeffreys, J. F. Y. Brookfield, and R. Semeonoff, “Positive identification of an immigration test-case using human DNA fingerprints,” Nature, vol. 317, pp. 818-819, 1985; K. Inman and N. Rudin, An Introduction to Forensic DNA Analysis. Boca Raton, Fla.: CRC Press, 1997), incorporated by reference, and has application to criminal justice.

The parallel detection feature of the transform-based sequencing method specified herein is ideally suited for these applications. By partially characterizing the alleles of a standardized set of polymorphic loci of many individuals at high-throughput, large populations can be genetically fingerprinted for low cost, effort, and time. In one preferred embodiment of the invention for this use, the Laplace transform experiment for genotyping tandem repeat length polymorphisms is done using a standard reference set, such as the SGMplus muliplex set (i.e., the forensic markers D3, VWA, D16, D2, AMELO, D8, D21, D18, D19, THO1, and FGA). In the most preferred embodiment for high-throughput data generation, multiplex PCR products of individuals are placed onto surfaces, and the Laplace transform-based sequencing is performed on the surfaces. This embodiment enables ultra-high-throughput data generation for database formation or casework. Alternatively, the locus detection sequences can be placed on a surface, and used as a hybridization capture target for a labeled transform-sequencing probe.

### Positional Cloning

In the positional cloning of genes, standard steps include: (a) screening the genomes of related individuals with polymorphic markers to determine the location(s) of the genes related to the phenotype of interest, (b) performing mutation analysis on some individuals to identify the causative gene, and (c) sequencing the gene region. This has been well described (D. Cohen, I. Chumakov, and J. Weissenbach, Nature, vol. 366, pp. 698-701, 1993; B.-S. Kerem, J. M. Rommens, J. A. Buchanan, D. Markiewicz, T. K. Cox, A. Chakravarti, M. Buchwald, and L.-C. Tsui, “Identification of the cystic fibrosis gene: genetic analysis,” Science, vol. 245, pp. 1073-1080, 1989; J. R. Riordan, J. M. Rommens, B.-S. Kerem, N. Alon, R. Rozmahel, Z. Grzelczak, J. Zielenski, S. Lok, N. Plavsic, J.-L. Chou, M. L. Drumm, M. C. Iannuzzi, F. S. Collins, and L.-C. Tsui, “Identification of the cystic fibrosis gene: cloning and characterization of complementary DNA,” Science, vol. 245, pp. 1066-1073, 1989), incorporated by reference.

The parallel detection feature of the transform-based sequencing method specified herein is ideally suited for all these applications. More specifically: (a) By partially characterizing the alleles of polymorphic loci of many individuals at high-throughput, large populations can be genotyped for low cost, effort, and time. One preferred embodiment of the invention is the Laplace transform for genotyping tandem repeat length polymorphisms. (b) The mutation analysis is done by partially characterizing the gene sequences. One preferred embodiment of the invention for this application is using the Laplace transform for obtaining distinguishing partial sequence signatures. (c) Sequencing the entire gene region is preferrably done using the invention.

### Expression Analysis

Only a subset of genes are switched on in a given cell. This gene expression state depends on the type of tissue, its disease state, and external modulations (e.g., pharmacological agents and other environmental factors). Associating a gene expression profile with a tissue state can help identify causative genes that lead to that tissue state.

Massively parallel DNA sequencing for gene expression can be done using the transform-based sequencing invention. In one preferred embodiment, this is accomplished using an EST-profiling method (M. D. Adams, J. M. Kelley, J. D. Gocayne, M. Dubnick, M. H. Polymeropoulos, H. Xiao, C. R. Merril, A. Wu, B. Olde, R. F. Moreno, A. R. Kerlavage, W. R. McCombie, and J. C. Venter, “Complementary DNA sequencing: Expressed sequence tags and human genome project,” Science, vol. 252, pp. 1651-1656, 1991), incorporated by reference.

The cDNA sequencing tempates are prepared from the tissue as in the standard EST method. However, instead of individually sequencing each template by Sanger sequencing and gel electrophoresis, the templates are deposited onto two dimensional surfaces and the parallel labeled-synthesis transform sequencing method is applied, as described herein. One distinguishing feature of the invention relative to the prior art is the ten to thousand-fold increase in parallelization of DNA sequencing templates when using very small zero-dimensional spots on a two dimensional surface, instead of the more space-consuming sets of one-dimensional lanes or runs.

### Cancer Monitoring

DNA sequencing is performed to study cancer cells. Transform-based DNA sequencing can be used to characterize chromosomal DNA, or the mRNA (usually in cDNA form) of expressed genes. Such molecular analyses of sample tissues are useful in prevention, diagnosis, staging, assessment, and treatment in the cancer management process. Molecular characterization also enables detailed study of cancer pathogenesis, which can lead to an understanding of the disease mechanism and (ultimately) cures or other treatments. Moreover, the genotyping transform-based sequencing method described herein is applicable to cancer monitoring.

Somatic deletions of chromosomal regions that contain tumor suppressor genes are helpful in mapping tumor-specific genes and in monitoring patients with specific tumors. These somatic deletions can be detected as a loss of heterozygosity (LOH) through genetic (e.g., microsatellite) analysis of tumor tissues (F. Canzian, A. Salovaara, P. Kristo, R. B. Chadwick, L. A. Aaltonen, and A. de la Chapelle, “Semiautomated assessment of loss of heterozygosity and replication error in tumors,” Cancer Research, vol. 56, pp. 3331-3337, 1996), incorporated by reference. The STR genotyping transform-based sequencing method described herein is applicable to monitoring LOH.

Mismatch repair genes help eliminate PCR stutter errors during DNA replication. Defects in these DNA repair genes can be detected via microsatellite instability (MI). MI is a change in allele length polymorphism in a tumor relative to normal tissue; MI is also called replication error (RER) (S. Thibodeau, G. Bren, and D. Schaid, “Microsatellite instability in cancer of the proximal colon,” Science, vol. 260, no. 5109, pp. 816-819, 1993; L. Cawkwell, L. Ding, F. A. Lewis, I. Martin, M. F. Dixon, and P. Quirke, “Microsatellite instability in colorectal cancer: improved assessment using fluorescent polymerase chain reaction,” Gastroenterology, vol. 109, pp. 465-471, 1995), incorporated by reference. The STR genotyping transform-based sequencing method described herein is applicable to monitoring MI.

### Agriculture

DNA sequencing methods are used in agricultural studies, in both plant and animal science. For genetic linkage mapping, the parallel detection feature of the transform-based sequencing method specified herein is ideally suited for large-scale application of these genetic linkage maps on many animals. By partially characterizing the alleles of polymorphic loci of many animals at high-throughput, large populations can be studied for low cost, effort, and time. One preferred embodiment uses the Laplace transform for genotyping tandem repeat length polymorphisms. Large-scale genetic linkage maps of polymorphic DNA markers exist for many species (W. Barendse, D. Vaiman, S. J. Kemp, Y. Sugimoto, S. M. Armitage, J. L. Williams, H. S. Sun, A. Eggen, M. Agaba, S. A. Aleyasin, M. Band, M. D. Bishop, J. Buitkamp, K. Byrne, F. Collins, L. Cooper, W. Coppettiers, B. Denys, R. D. Drinkwater, K. Easterday, C. Elduque, S. Ennis, G. Ehrhardt, L. Ferretti, and P. Zaragoza, “A medium-density genetic linkage map of the bovine genome,” Mamm. Genome, vol. 8, no. 1, pp. 21-28, 1997; H. H. Cheng, “Mapping the chicken genome,” Poult. Sci., vol. 76, no. 8, pp. 1101-1107, 1997; S. M. Kappes, J. W. Keele, R. T. Stone, R. A. McGraw, T. S. Sonstegard, T. P. Smith, N. L. Lopez-Corrales, and C. W. Beattie, “A second-generation linkage map of the bovine genome,” Genome Res., vol. 7, no. 3, pp. 235-249, 1997; G. A. Rohrer, L. J. Alexander, Z. Hu, T. P. Smith, J. W. Keele, and C. W. Beattie, “A comprehensive map of the porcine genome,” Genome Res., vol. 6, no. 5, pp. 371-391, 1996), incorporated by reference.

Another application of the transform sequencing invention is for quantitative trait determination for genetically improving crop and livestock species. In the most preferred embodiment, a Laplace transform is used to genotype tandem repeat length polymorphisms on large two dimensional arrays of individual DNAs. Quantitative traits are used effectively in the current agricultural art (M. Georges, D. Nielson, M. Mackinnon, A. Mishra, R. Okimoto, A. T. Pasquino, L. S. Sargeant, A. Sorensen, M. R. Steele, and X. Zhao, “Mapping quantitative trait loci controlling milk production in dairy cattle by exploiting progeny testing,” Genetics, vol. 139, no. 2, pp. 907-920, 1995; J. Hillel, “Map-based quantitative trait locus identification,” Poult. Sci., vol. 76, no. 8, pp. 1115-1120, 1997; R. J. Spielman, W. Coppieters, L. Karim, J. A. van Arendonk, and H. Bovenhuis, “Quantitative trait loci analysis for five milk production traits on chromosome six in the Dutch Holstein-Friesian population,” Genetics, vol. 144, no. 4, pp. 1799-1808, 1996), incorporated by reference.

Another application of the invention is for genetic risk assessment for crop or livestock disease. Such assessments can focus pharmacological treatments (prospectively or retrospectively) on at-risk plant or animals. These methods typically begin with determining genes that are linked to specific diseases. Once the genes have been found, the most preferred embodiment of the transform-based DNA sequencing methods specified herein would place amplified individual DNA of genome loci as spots onto multiple copies of a two dimensional surface, with each spot corresponding to an individual. Transform-based sequencing then obtains the partial sequence information about the m variations that distinguish the gene alleles, without requiring a complete sequence determination. Genetic risk assessment uses are well described in the current art (J. Hu, N. Bumstead, P. Barrow, G. Sebastiani, L. Olien, K. Morgan, and D. Malo, “Resistance to salmonellosis in the chicken is linked to NRAMP1 and TNC,” Genome Res., vol. 7, no. 7, pp. 693-704, 1997), incorporated by reference.

### Structure/Function

The sequence of a gene can be determined by the transform-based DNA sequencing method. From this gene sequence, the relation of a gene or its promoters to other known functions may be determined using similarity or homology searches. Protocols for these determinations are well described (N. J. Dracopoli, J. L. Haines, B. R. Korf, C. C. Morton, C. E. Seidman, J. G. Seidman, D. T. Moir, and D. Smith, ed., Current Protocols in Human Genetics. New York: John Wiley and Sons, 1999), incorporated by reference. The use of expressed sequence tag (EST) databases (Merck Gene Index, St. Louis, Mo.; Human Genome Sciences, Gathersburg, Md.) together with the genome sequence provides a highly effective means for rapidly correlating a gene's sequence with the structure and function of its protein products.

### Sequencing System

The invention includes a system for nucleic acid sequencing comprising (a) a means for amplifying a nucleic acid sample to produce an amplified nucleic acid product; (b) a means for extending a sequencing primer bound to the DNA product in the presence of terminating nucleotide analogs to produce a collection of labeled nucleic acid products, said extending means in connection with the amplified product; (c) a means for detecting a total amount of label present in the collection to produce a measurement, said detecting means in connection with the collection; and (d) a means for combining a plurality of measurements to determine DNA sequence information about the sample, said combining means in connection with the measurement.

In a most preferred embodiment, the amplifying means includes a PCR thermocycler, the extending means includes a chamber that permits DNA sequencing reactions to occur in the presence of terminating nucleotide analogs, the detecting means measures fluorescent or other labels that quantify an amount of DNA molecules, and the combining means includes a computing device with memory.

### Inducing Decay

In general terms, the invention provides a mechanism for inducing a decay function, and imposing said decay function on an unknown signal. When said induced decay is imposed on the signal, a numerical quantity is formed which characterizes the signal's behavior in the presence of the decay function. By combining a plurity of such numerical quantities, information is obtained about the signal. In one preferred embodiment, the unknown signal is a nucleic acid sequence, the decay function is induced by introducing dideoxy terminator analogs into a sequencing reaction, the numerical quantities correspond to Laplace transform coefficients, and the obtained information serves to characterize the sequence. Complete characterization is not essential in many useful applications, such as detecting genetic polymorphism.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

What is claimed is:

1. A method of nucleic acid sequencing comprising the steps:
(a) amplifying a nucleic acid sample to produce an amplified DNA product;
(b) extending a sequencing primer bound to the DNA product in the presence of terminating nucleotide analogs to produce a collection of labeled nucleic acid products;
(c) detecting a total amount of label present in the collection to produce a measurement; and
(d) combining a plurality of measurements to determine DNA sequence information about the sample.

2. A method as described in claim 1 wherein each measurement of a label corresponds to an amount of terminating nucleotide.

3. A method as described in claim 1 wherein the DNA sequence information corresponds to a length of the DNA sequence.

4. A method as described in claim 1 wherein the DNA sequence information corresponds to a plurality of bases in the DNA sequence.

5. A method as described in claim 1, wherein after the combining step, the DNA sequence information is used for human identification.

6. A method as described in claim 1, wherein after the combining step, the DNA sequence information is used for diagnostic testing.

7. A method as described in claim 1, wherein after the combining step, the DNA sequence information is used for genetic localization or gene discovery.

8. A method as described in claim 1, wherein after the combining step, the DNA sequence information is used for criminal justice applications.

9. A method as described in claim 1, wherein after the combining step, the DNA sequence information is used in conjunction with a DNA database of genetic polymorphisms.

10. A method as described in claim 1, wherein after the combining step, the DNA sequence information is used for cancer assessment.

11. A system for nucleic acid sequencing comprising:
(a) a means for amplifying a nucleic acid sample to produce an amplified nucleic acid product;
(b) a means for extending a sequencing primer bound to the DNA product in the presence of terminating nucleotide analogs to produce a collection of labeled nucleic acid products, said extending means in connection with the amplified product;
(c) a means for detecting a total amount of label present in the collection to produce a measurement, said detecting means in connection with the collection; and
(d) a means for combining a plurality of measurements to determine DNA sequence information about the sample, said combining means in connection with the measurement.

12. A system as described in claim 11, wherein the amplifying means includes a PCR thermocycler, the extending means includes a chamber that permits DNA sequencing reactions to occur in the presence of terminating nucleotide analogs, the detecting means measures fluorescent or other labels that quantify an amount of DNA molecules, and the combining means includes a computing device with memory.

13. A method for obtaining information about a signal comprising the steps:
(a) inducing a decay function;
(b) imposing the decay function on a signal;
(c) forming a numerical quantity that characterizes the signal's behavior in the presence of the decay function;
(d) combining a plurity of such numerical quantities to obtain information about the signal.

14. A method as described in claim 13 wherein the signal is a nucleic acid sequence, the decay function is induced by introducing dideoxy terminator analogs into a sequencing reaction, the numerical quantities correspond to Laplace transform coefficients, and the obtained information helps characterize the sequence.

15. A method as described in claim 14 wherein the characterization does not completely describe the nucleic acid sequence.

16. A method as described in claim 15 wherein the incomplete sequence information describes a genetic polymorphism.
